(19) United States
(12) Patent Application Publication
(10) Pub. No.: US 2015/0197785 A1
(43) Pub. Date: Jul. 16, 2015

(54) METHODS AND APPARATUS FOR ANALYZING AND QUANTIFYING DNA ALTERATIONS IN CANCER

(71) Applicants: The Broad Institute, Inc., Cambridge, MA (US); Dana-Farber Cancer Institute, Inc., Boston, MA (US)

(72) Inventors: Scott L. Carter, Cambridge, MA (US); Gad Getz, Belmont, MA (US); Aaron McKenna, Essex Junction, VT (US); Matthew Meyerson, Concord, MA (US)

(73) Assignees: The Broad Institute, Inc., Cambridge, MA (US); Dana-Farber Cancer Institute, Inc., Boston, MA (US)

(21) Appl. No.: 14/420,750

(22) PCT Filed: Aug. 9, 2013
(86) PCT No.: PCT/US13/54310

§ 371 Date: Feb. 10, 2015

Related U.S. Application Data
(60) Provisional application No. 61/681,694, filed on Aug. 10, 2012.

(51) Int. Cl. C12Q 1/68

(52) U.S. Cl. CPC C12Q 1/68

(57) ABSTRACT

Methods and apparatus for inferring purity and ploidy from a sample of cells (e.g., a sample comprising cancer and normal cells) are described. Copy number per cell of interest (e.g., cancer cell) is determined by optimizing purity and ploidy for the sample based, at least in part, on relative copy number profile information. One or more likelihood fit scores are determined for each of a plurality of candidate solutions generated by the methods described herein. A solution is selected based, at least in part on the likelihood fit score(s) and the copy number per cancer cell is determined in accordance with the selected solution.

### RELATED APPLICATIONS

This application claims priority to U.S. Provisional Application Ser. No. 61/681,694 filed Aug. 10, 2012, which is incorporated by reference in its entirety.

### FEDERALLY SPONSORED RESEARCH

This invention was made with government support under U24CA126546 awarded by the National Institutes of Health, U24CA143867 awarded by the National Institutes of Health, and U24CA143845 awarded by the National Institutes of Health. The government has certain rights in the invention.

### BACKGROUND

Understanding the structure and evolution of somatic alterations in the cancer genome is important for developing targeted therapies for cancer patients. Conventional genomic characterization techniques measure somatic alterations in a cancer sample in units of genomes (DNA mass). Measuring somatic copy-number alterations (SCNAs) on a relative basis using well-known techniques such as microarrays or massively parallel sequencing technology has been the standard approach for copy-number analysis since the development of comparative genomic hybridization (CGH).

### SUMMARY

Some embodiments of the invention are directed to methods and apparatus for jointly estimating sample (e.g., tumor sample) purity and ploidy to infer information about somatic copy number per cancer cell, which provides advantages over conventional techniques relying on relative measurements of SCNAs. Additionally, some embodiments are directed to inferring the multiplicity of somatic mutations in integer allelic units per cancer cell to enable the characterization of mutations as clonal or subclonal. In some embodiments, such mutations are point mutations. Such a classification of mutations allows for the further study of the clonal evolution in different cancer types to improve an understanding of the time sequence of different markers for cancer development and to provide insight into possible targeted therapeutic cancer treatment regimes.

Some embodiments are directed to a method of determining a copy number per cancer cell in a sample of cells. The method comprises receiving a relative copy number profile for DNA segments extracted from the sample; determining based, at least in part, on the relative copy number profile, a set of candidate solutions by estimating purity and ploidy from information about somatic copy number alterations in the sample; determining a likelihood fit score for each of the solutions in the set of candidate solutions, wherein the likelihood fit score is determined based, at least in part, on the information about somatic copy number alterations in the sample and information about one or more mutations detected in the sample; selecting a solution from the set of candidate solutions based, at least in part, on the likelihood fit score associated with each candidate solution; and determining the copy number per cancer cell in accordance with the selected solution.

Other embodiments are directed to a computer-readable medium encoded with a plurality of instructions that, when executed by a computer, perform a method, comprising: receiving a relative copy number profile for DNA segments extracted from the sample; determining based, at least in part, on the relative copy number profile, a set of candidate solutions by estimating purity and ploidy from information about somatic copy number alterations in the sample; determining a likelihood fit score for each of the solutions in the set of candidate solutions, wherein the likelihood fit score is determined based, at least in part, on the information about somatic copy number alterations in the sample and information about one or more mutations detected in the sample; selecting a solution from the set of candidate solutions based, at least in part, on the likelihood fit score associated with each candidate solution; and determining the copy number per cancer cell in accordance with the selected solution.

Other embodiments are directed to a computer system, comprising: at least one processor programmed to: at least one processor programmed to: receive a relative copy number profile for DNA segments extracted from the sample; determine based, at least in part, on the relative copy number profile, a set of candidate solutions by estimating purity and ploidy from information about somatic copy number alterations in the sample; determine a likelihood fit score for each of the solutions in the set of candidate solutions, wherein the likelihood fit score is determined based, at least in part, on the information about somatic copy number alterations in the sample and information about one or more mutations detected in the sample; select a solution from the set of candidate solutions based, at least in part, on the likelihood fit score associated with each candidate solution; and determine the copy number per cancer cell in accordance with the selected solution.

Other embodiments are directed to a method of determining a copy number per cancer cell in a sample, the method comprising: receiving a relative copy number profile for DNA segments extracted from the sample; determining based, at least in part, on the relative copy number profile, a set of candidate solutions by estimating purity and ploidy from information about somatic copy number alterations in the sample; determining a likelihood fit score for each of the solutions in the set of candidate solutions, wherein the likelihood fit score is determined based, at least in part, on the information about somatic copy number alterations in the sample and information about karyotype copy profile characteristics of a particular disease; selecting a solution from the set of candidate solutions based, at least in part, on the likelihood fit score associated with each candidate solution; and determining the copy number per cancer cell in accordance with the selected solution.

Other embodiments are directed to a method of classifying a mutation in a DNA sample as clonal or subclonal, the method comprising: determining a cancer cell fraction for the mutation; and classifying the mutation as clonal in response to determining that there is a greater than 50% probability that the mutation exists in a cancer cell fraction below a threshold value.

Other embodiments are directed to a method of determining a read depth sufficient to detect a mutation in a sample, the method comprising: receiving an estimate of purity and copy number per cancer cell; determining the read depth based, at least in part, on the estimate of purity and the estimate of copy number per cancer cell.

Other embodiments are directed to a method of determining a power estimate for a given read depth needed to detect a mutation in a sample, the method comprising: determining the power estimate based, at least in part, on an estimate of the purity of the sample, a cancer cell fraction for the mutation, and a copy number per cell estimate.

Other embodiments are directed to a method of evaluating a somatic evolution of a mutation in a cancer genome, the method comprising: determining a first cancer cell fraction for the mutation at a first timepoint; determining a second cancer cell fraction for the mutation at a second timepoint; and evaluating the somatic evolution of the mutation by comparing the first cancer cell fraction and the second cancer cell fraction.

It should be appreciated that all combinations of the foregoing concepts and additional concepts discussed in greater detail below (provided that such concepts are not mutually inconsistent) are contemplated as being part of the inventive subject matter disclosed herein.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 is an exemplary process for determining copy number per cell in a sample of cancer cells and normal cells in accordance with some embodiments of the invention;

FIG. 2 is an exemplary process for analyzing a sample in accordance with some embodiments of the invention;

FIG. 3 is an example of a relative copy number profile used in accordance with some embodiments of the invention;

FIGS. 4A-4C are examples of karyotype information used in accordance with some embodiments of the invention;

FIG. 5 is an example of a set of candidate solutions used to determine absolute somatic copy numbers in accordance with some embodiments of the invention;

FIG. 6 illustrates purity/ploidy values and corresponding exemplary likelihood fit scores used, at least in part, to select a solution from the set of candidate solutions, in accordance with some embodiments of the invention;

FIG. 7 is an exemplary computer system on which some embodiments of the invention may be employed; and

FIGS. 8A-8C illustrate results of a longitudinal analysis of genetic evolution in cancer performed as a validation of at least some of the techniques described herein.

### DETAILED DESCRIPTION

Interpreting and comparing relative copy number measurements across samples is complicated because such measurements are dependent on a sample's purity and its overall ploidy. To reduce this complexity, the inventors have recognized and appreciated that measuring the number of copies per cancer cell (e.g., absolute copy number), rather than relative copy numbers, is beneficial because such measurements are straightforward to interpret and, for alterations that are fixed in the cancer cell population, are simple integer values.

The inventors have also recognized that measuring copy number per cancer cell using known methods is considerably more challenging than measuring relative copy number in units of diploid DNA mass in a sample for several reasons. For example, cancer cells are nearly always intermixed with an unknown fraction of normal cells (purity), the actual DNA content of the cancer cells (ploidy), resulting from gross numerical and structural chromosomal abnormalities, is unknown, and the cancer cell population may be heterogeneous, perhaps owing to ongoing subclonal evolution. In principle, one could infer copy number per cell by rescaling relative data on the basis of cytological measurements of DNA mass per cancer cell, or by single-cell sequencing approaches. However, such approaches are not suited to support initial large-scale efforts to comprehensively characterize the cancer genome or ongoing efforts to provide meaningful clinical sequencing of patient samples to identify, monitor and adjust therapeutic strategies all of which require the ability to assess and control for sample purity and also to understand evolution of subclonal populations. To this end, some embodiments of the invention are directed to methods and apparatus for characterizing somatic DNA alterations on a cellular basis to provide a foundation for integrative genomic analysis of the cancer genome. By correlating purity and ploidy estimates of a sample of cancer and normal cells with expression subtypes, statistical power calculations may be developed and used to select well-powered samples for whole-genome sequencing to analyze different types of cancer populations.

Some embodiments are directed to the development of a reliable, high-throughput method to infer cellular homologous copy numbers from DNA samples, as well as multiplicity values of mutations. Sample purity and ploidy are estimated directly from a relative copy profile for a sample containing a mixture of cancer cells and normal cells. The relative copy profile may be determined in any suitable way including, but not limited to, the methods described above or by using alternate methods such as whole-exome sequencing. In practice, estimation of purity and ploidy may not be fully determined on a single sample leading to multiple candidate solutions. In some embodiments, information related to exemplary karyotypes for particular disease types may be used to help resolve multiple candidate solutions, as discussed in more detail below. Additionally, some embodiments use information about somatic mutations to select a solution from the set of candidate solutions. For example, the information about somatic point mutations may be obtained using whole-exome sequencing which identifies somatic single nucleotide variations (SSNVs). In such embodiments, the multiplicity of somatic point-mutations in integer allelic units per cancer cell may be inferred. In some embodiments, both the relative copy number profile and the information about one or more mutations may be determined using massively parallel sequencing techniques including, but not limited to, whole-genome sequencing and whole-exome sequencing. Methods for determining a relative copy number profile using whole-exome sequencing are known. For example, a relative copy number profile may be determined by read depth.

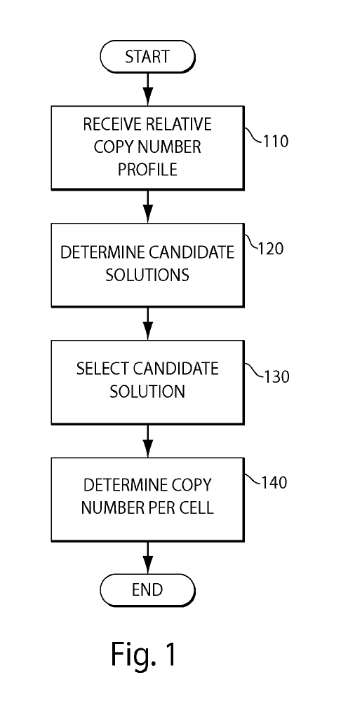

FIG. 1 illustrates an exemplary process for determining copy number per cell in accordance with some embodiments of the invention. When DNA is extracted from a sample containing a mixed population of cancer and normal cells, information about copy number per cancer cell is lost. Some embodiments of the invention infer this information from the population of mixed DNA by analyzing relative copy-number profile information for DNA segments in the sample. As shown in the exemplary process of FIG. 1, in act 110, relative copy number profile information is received. In some embodiments, the relative copy number profile information may be generated by processing a sample using microarrays, massively parallel sequencing (including whole-genome sequencing or whole-exome sequencing) or some other suitable method. In act 120, the received relative copy number profile information may be analyzed to determine one or more candidate solutions representing likely combinations of purity and ploidy. Methods for estimating purity and ploidy in accordance with some embodiments of the invention are described in more detail below. In act 130, one or more information sources may be used to select one of the candidate solutions based, at least in part, on likelihood fit scores associated with the candidate solutions. For example, as described in more detail below, the one or more information sources may include, but are not limited to, precomputed models of recurrent cancer karyotypes and allelic fraction values for somatic point mutations. In act 140, the selected candidate solution may be used to determine, among other things, the cellular copy number the sample.

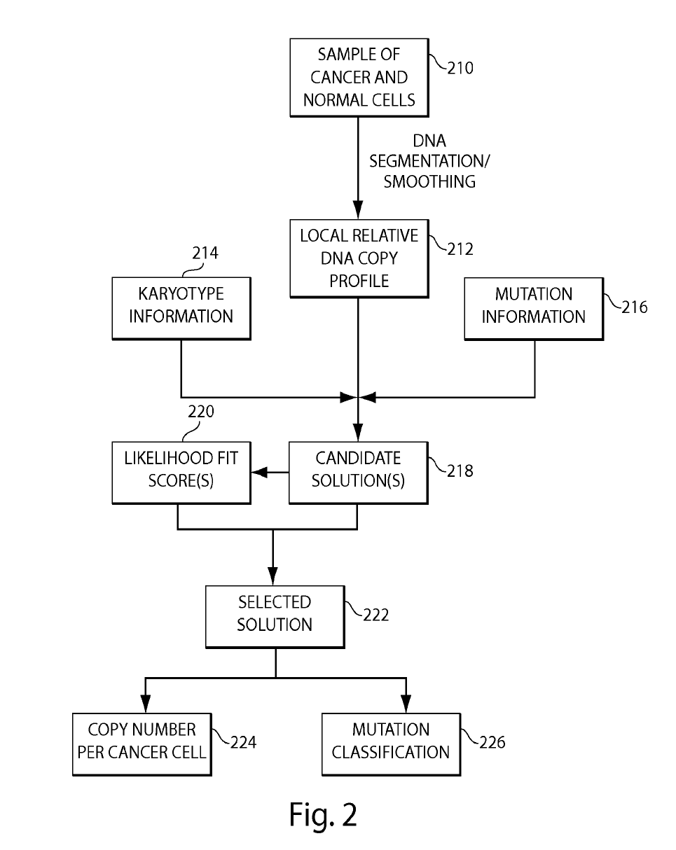

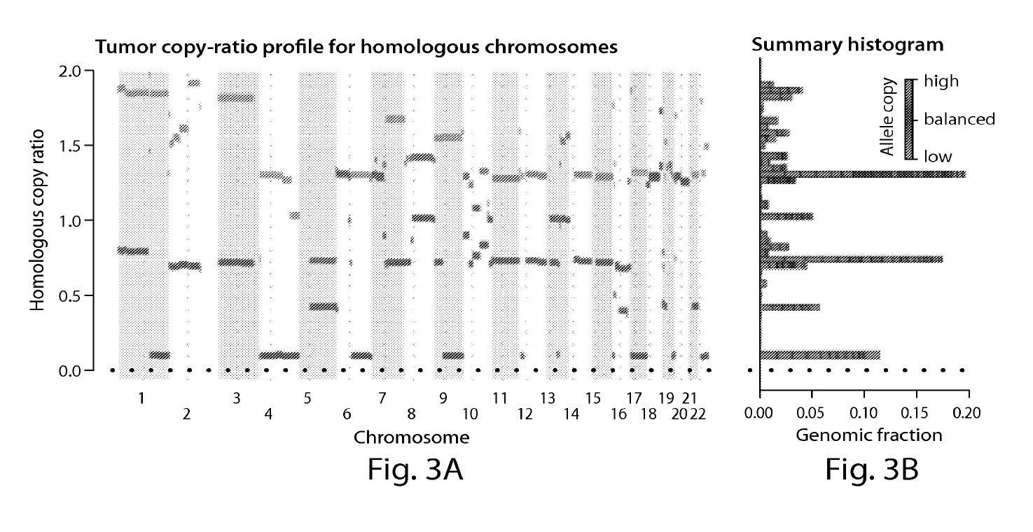

FIG. 2 illustrates a flow chart of an exemplary process for analyzing samples in accordance with some embodiments of the invention. In act 210, a sample having a mixture of cancer and normal cells is provided as input for analysis. The mixture of cells is processed using DNA segmentation and smoothing and the process proceeds to act 212 where the segmented data is used to determine a local relative DNA copy profile. An example of a local relative DNA copy profile and a corresponding summary histogram is illustrated in FIG. 3. The local relative DNA copy profile is used together with precomputed karyotype information 214 and mutation information 216 (e.g., point mutation information) to determine one or more candidate solutions for purity and ploidy, as discussed in more detail below.

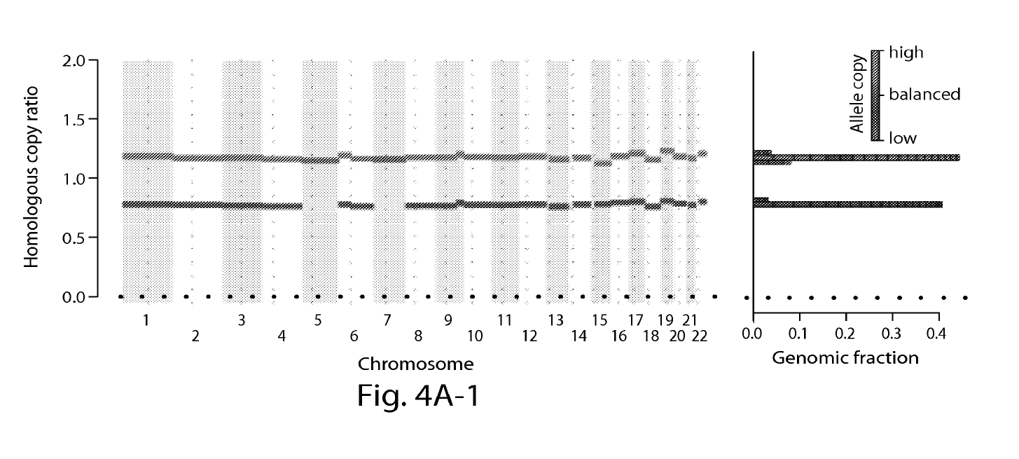

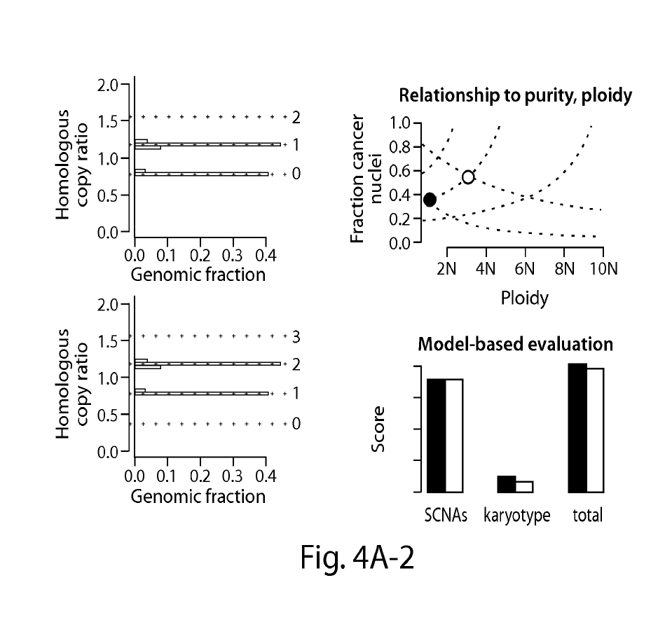

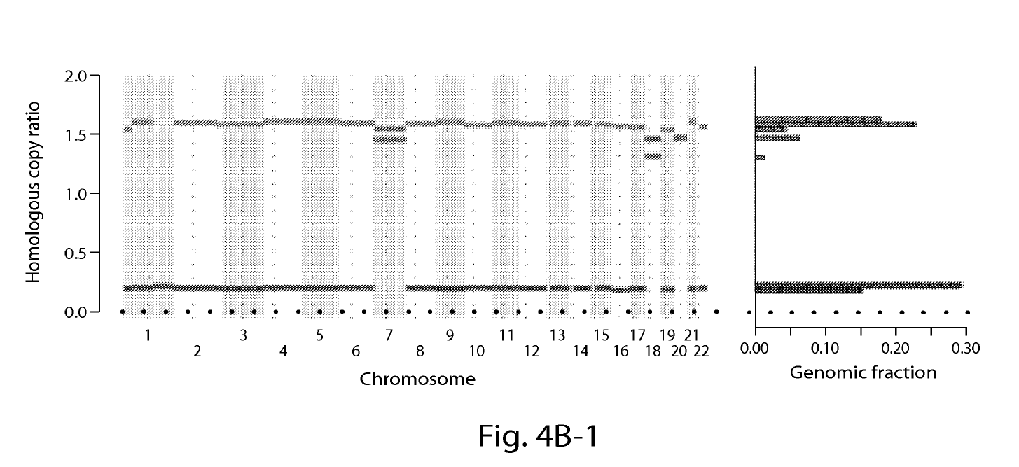

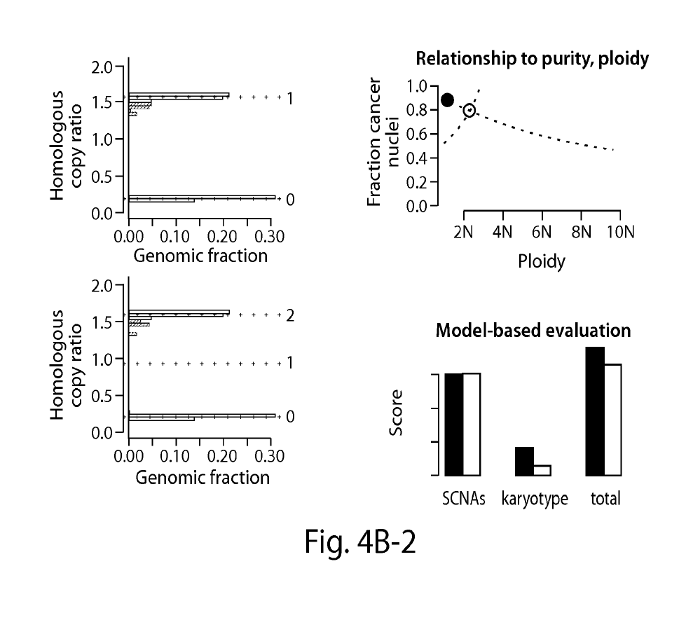

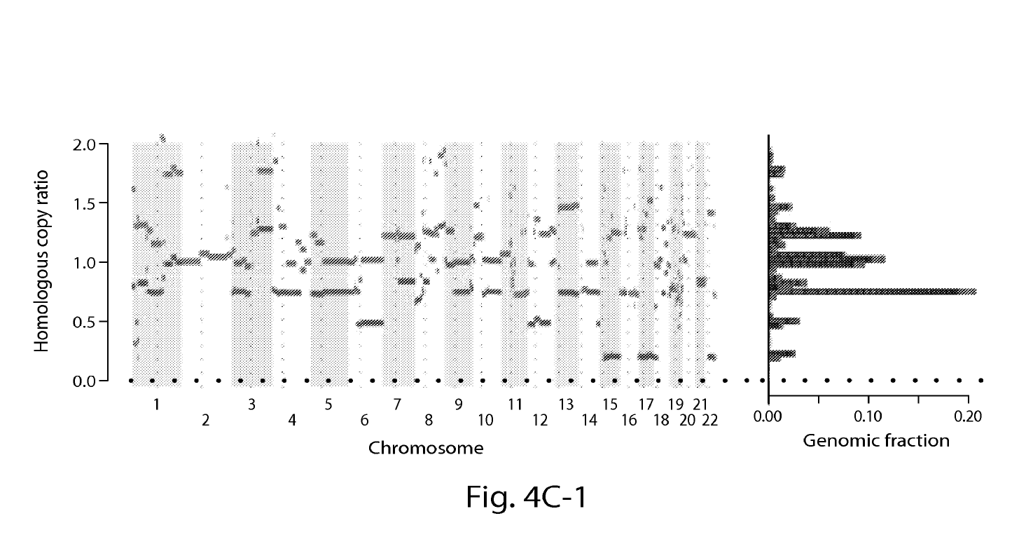

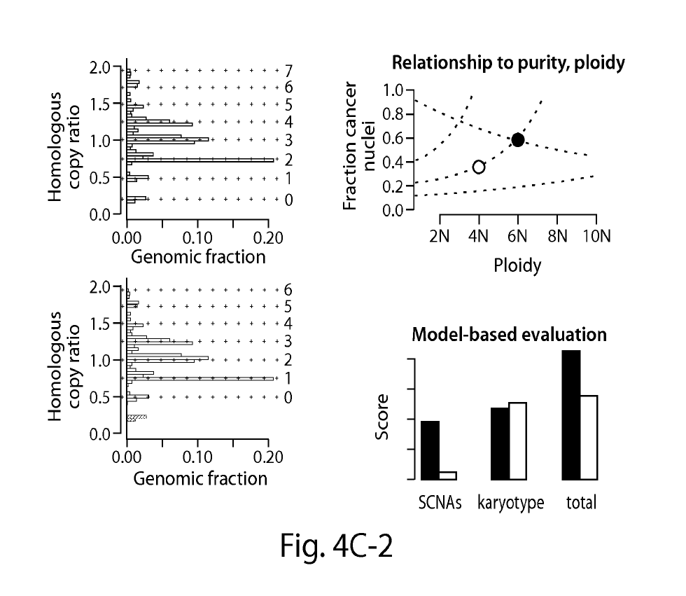

FIGS. 4A-C illustrate exemplary karyotype information that may be used in accordance with some embodiments of the invention. FIG. 4A illustrates analysis of a lung adenocarcinoma sample SM-11ZY with near haploid genomes (purity=0.36, ploidy=1.12), FIG. 4B illustrates analysis of a glioma sample ‘glioma 612’ with hyperaneuploid genomes (purity=0.88, ploidy=1.14), and FIG. 4C illustrates analysis of a HGS-OvCa sample TCGA-25-1320-01A-01D-0452-01 having hyperaneuploid genomes (purity=0.58, ploidy=6.03). As discussed in more detail below, in some embodiments, a set of karyotype information may be used as prior information in a probabilistic model to jointly estimate purity and ploidy, where the function of the karyotype information is to constrain the number of possible candidate solutions by eliminating possible solutions inconsistent with the precomputed karyotype information.

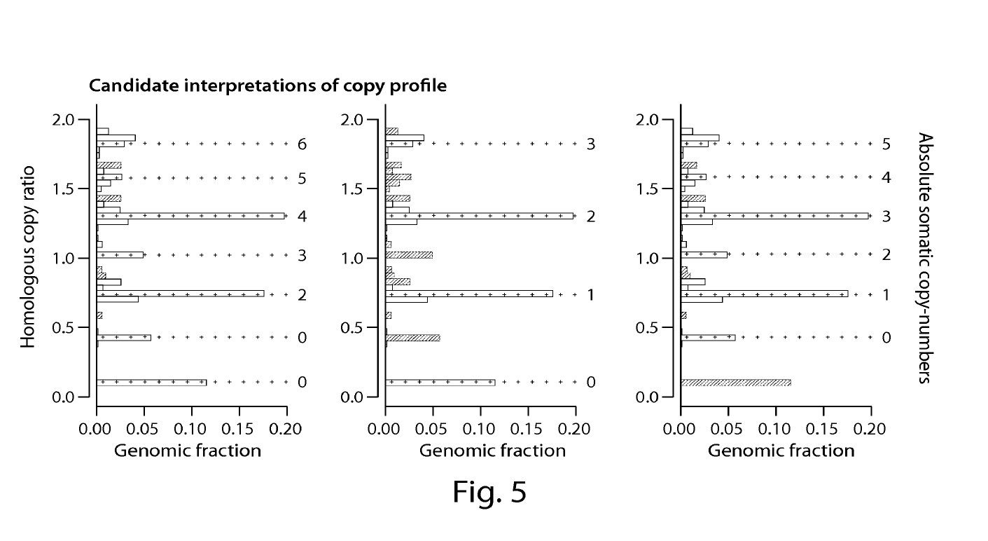

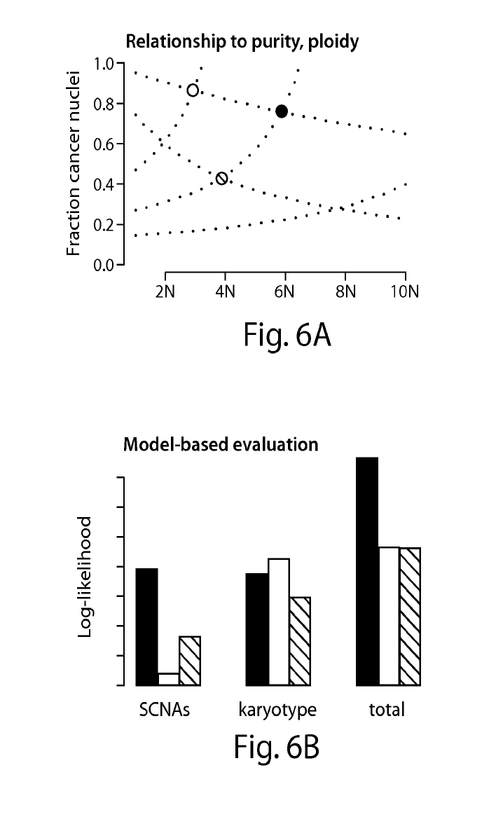

FIG. 5 illustrates an example of multiple candidate solutions for cellular somatic copy-number profiles generated in act 218, in accordance with some embodiments of the invention. The local minima identified for the purity/ploidy values for each of the candidate solutions may also be represented as illustrated in FIG. 6, and likelihood fit scores 220 may be determined for each of the solutions to facilitate the selection of one of the candidate solutions in the set. Although only SCNA- and karyotype-fit scores are illustrated in FIG. 6B, it should be appreciated that other likelihood fit scores may also be used. For example, in embodiments that determine the set of candidate solutions based, at least in part, on somatic point mutation information, a mutation likelihood fit score may also be determined, and this additional score may be used, at least in part, to select a solution. As illustrated in FIG. 6B, in some embodiments, a total (i.e., combined) likelihood score may be determined based, at least in part, on one or more of the individual likelihood scores and the total likelihood score may be determined using any suitable weighting factors for combining two or more of the individual likelihood scores.

In response to determining the one or more likelihood fit scores, a solution is selected in act 222 based, at least in part, on the likelihood fit score(s). For example, as illustrated in FIG. 6B, the candidate solution having a ploidy of 6N and a purity of 0.8 has the highest total likelihood fit score despite having the second highest karyotype fit score. In some embodiments, the candidate solution with the greatest total likelihood fit score may be determined as the selected solution in act 222. As discussed in more detail below, the selected solution may be used to determine, among other things, copy number per cancer cell 224 and mutation classification 226 (e.g., clonal or subclonal).

An illustrative example for processing samples in accordance with some embodiments of the invention is now described. Consider a cancer-tissue sample including a mixture of a proportion a of cancer cells (assumed to be monogenomic—that is, with homogenous SCNAs in the cancer cells) and a proportion (1−α) of contaminating normal (diploid) cells. For each locus x in the genome, let q(x) denote the integer copy number of the locus in the cancer cells and let τ denote the mean ploidy of the cancer-cell fraction, defined as the average value of q(x) across the genome. In the mixed cancer sample, the average absolute copy number of locus x is αq(x)+2(1−α) and the average ploidy (D) is ατ+2(1−α), measured in units of haploid genomes.

The relative copy number (R) of locus x is therefore:

R(x)=(αq(x)+2(1−α))/D=(α/D)q(x)+(2(1−α)/D) (1)

Because q(x) takes integer values, R(x) takes discrete values. The smallest possible value is (2(1−α)/D), which occurs at homozygously deleted loci and corresponds to the fraction of DNA from normal cells. The spacing between values (α/D) corresponds to the concentration ratio of alleles present at one copy per cancer cell and zero copies per normal cell. Notably, if a cancer sample is not strictly clonal, copy-number alterations occurring in substantial subclonal fractions will appear as outliers from this pattern.

As discussed above, in some embodiments, the cellular copy inference may be extended to encompass somatic mutations (e.g., point mutations) as follows:

F(x)=(αs_{d}(x))/Dₛ=(α/Dₛ)s_{q}(x), (2)

where, s_{q} represents the multiplicity of the mutation in integer values per cancer cell (which cannot exceed q(x)), and Dₛ=αq(x)+2(1−α). The values of F(x) correspond to the expected fraction of sequencing reads that support the mutation, which depend on the sample purity and cellular somatic copy number at the mutant locus, q(x).

As discussed above, some embodiments examine possible mappings from relative to integer copy numbers by jointly optimizing the two parameters representing purity (α) and ploidy (τ). In some cases, several such mappings are possible, corresponding to multiple optima. The identification of candidate purity and ploidy values in accordance with some embodiments of the invention is discussed below.

In some embodiments, input homologue-specific copy ratio (HSCR) estimates are fit with a Gaussian mixture model, with components centered at the discrete concentration-ratios in accordance with equation (1) above. Although HSCR estimates are discussed in the example that follows, it should be appreciated that total-copy ratio data (e.g., from array CGH or low-pass sequencing data) may alternatively be used. The model used to fit the HSCR estimates may also support a moderate fraction of subclonal events which are not restricted to the discrete levels associated with integer values. A set of candidate solutions may be identified by searching for local optima of this likelihood over a range of purity and ploidy values. Each of the candidate solutions in the set may be associated with one or more likelihood fit scores to facilitate the selection of one of the solutions from the set. For example, each of the candidate solutions may be associated with a corresponding SCNA-fit likelihood score, which quantifies the evidence for each solution contributed by explanation of the observed HSCRs as integer SCNAs. In determining the SCNA-fit values, the input data may consist of N HSCRs xᵢ, iε{1, . . . , N}, with each of the HSCRs being observed with standard error σᵢ, and corresponding to a genomic fraction denoted wᵢ. Each of the xᵢ is assumed to have arisen from either one of Q integer copy-number states: Q={0, 1, . . . , Q−1}, or an additional state Z corresponding to a subclonal copy-number, as discussed above. The collection of possible copy-states may be defined as S=Q∪Z, where there are Q+1 indicators s for the copy-state of each segment, with p(sᵢ) representing the probability of segment i having been generated from state sεS. The integer copy-states of S are indexed by qεQ and the non-integer state is denoted by z.

The expected copy-ratio corresponding to each integer copy-number q(x) in a sample is given by equation (1), discussed above. When homologous copy-ratios are used, equation (1) becomes:

$${μ}_{q} = 2 [ \frac{α}{D} ] q ( x ) + [ \frac{2 ( α - 1 )}{D} ] \qquad ( 3 )$$

since HSCRs are measured relative to haploid concentrations, as opposed to the diploid values assumed by equation (1). D is related to purity and ploidy. The observed xᵢ may be modeled with a mixture of Q Gaussian components located at μ={μ_{q εQ}} representing integer copy-states Q and an additional uniform component Z. The mixture Z allows segments to be assigned non-integer copy values so that subclonal alterations or artifacts do not dramatically impact the likelihood.

$${s}_{i} Multinom ( p ( {s}_{i} \vert {w}_{i} , θ ) )$$

$${x}_{i} \{ \begin{matrix}{μ}_{q} + {∈}_{i} & if {s}_{i} ∈ Q \\ u & if {s}_{i} = Z\end{matrix} \} \\ {∈}_{i} N ( 0 , {σ}_{i}^{2} + {σ}_{H}^{2} )$$

$$u U ( d ) ,$$

N and Y denote the normal and uniform densities, respectively. The free parameter σ_{H} represents sample-level noise in excess of the HSCR standard-error σᵢ, which might represent a moderate number of related clones in the malignant cell population, ongoing genomic instability, or excessive noise due to variable experimental conditions. The mixture weights θ={θ_{sεS}} specify the expected genomic fraction allocated to each copy-state. The parameter d represents the domain of the uniform density, corresponding to the range of plausible copy-ratio values (e.g., d=7).

Because the data consist of copy-ratios calculated from a segmentation of the genome, the mixture weights P(si|wi, θ) should be calculated for each segment separately, taking into account the variable genomic fraction wᵢ. This may be accomplished by constraining the canonical averages of genomic mass allocated to each copy-state to match those specified by θ:

$${∀}_{s ∈ S} , {〈 {\sum }_{i = 1}^{N} {s}_{i} {w}_{i} 〉}_{C} = {θ}_{s} ,$$

where [symbol] • [symbol] _{C} denotes the average over all configurations {sᵢ}, weighted by the function C=P(sᵢ|wᵢ, λ). This density corresponds to the maximum entropy distribution over s subject to these constraints:

$$P ( {s}_{i} \vert {w}_{i} , λ ) = \frac{{e}^{- {λ}_{s} {s}^{\#} {w}_{i}}}{{{\sum }_{k ∈ {S}^{e - {λ}_{k} {k}^{\#} {w}_{i }}}}^{ } } ,$$

where s^{#} indicates the order of state s in a sequence of copy-states, beginning with 0. Values of the Q Lagrange multipliers λ are determined via Nelder-Mead optimization of L2 loss:

$$λ = arg {min}_{λ} {( {\sum }_{s ∈ S}^{ } [ {( {\sum }_{i = 1}^{N} [ {w}_{i} P ( {s}_{i} \vert {w}_{i} , λ ) ] - {θ}_{s} )}^{2} ] )}^{\frac{1}{2}}$$

This approximation allows for robustness of the SCNA-fit score to over-segmentation of the data. The likelihood of a given segment i is then calculated as:

$${L}_{p} ( {x}_{i} \vert μ , {σ}_{i} , {σ}_{H} , θ , {w}_{i} ) = {\sum }_{q ∈ Q}^{ } [ P ( {q}_{i} \vert {w}_{i} , λ ) N ( {x}_{i} \vert {μ}_{q} , {σ}_{i}^{2} + {σ}_{H}^{2} ) ] + P ( {z}_{i} \vert {w}_{i} , λ ) U ( d )$$

and the full log-likelihood of the data is then:

$${\sum }_{i = 1}^{N} log L ( {x}_{i} \vert μ , {σ}_{i} , {σ}_{H} , θ , {w}_{i} ) \qquad ( 5 )$$

The parameterization is defined as:

$$b = 2 ( 1 - α ) , {δ}_{τ} = \frac{α}{D}$$

which determines μ via equation (3). The set of candidate purity and ploidy solutions for a sample in accordance with some embodiments may be identified through optimization of equation (5) with respect to b and δ_{τ}. Calculation of equation (5) requires estimates of θ and σ_{H}, which are generally not known a priori. An approximation (scale-separation) may be made assuming that locations of the modes of equation (5) are invariant to moderate fluctuations in these parameters. A provisional likelihood for each xᵢ may then be calculated by:

$${L}_{p} ( {x}_{i} \vert μ , {σ}_{i} , σ ) = {\sum }_{q ∈ Q}^{ } [ N ( {x}_{i} \vert {μ}_{q} , {σ}_{i}^{2} + {σ}^{2} ) ] + U ( d )$$

Candidate purity and ploidy solutions may then be identified by optimization of:

$${\sum }_{i = 1}^{N} log L ( {x}_{i} \vert μ , {σ}_{i} , {σ}_{P} )$$

initiated from all points in a regular lattice spanning the domain of b and δ_{τ}. The parameter σ_{P} may be set to a suitable value (e.g., 0.01). Alternatively, the set of candidate solutions may be found using any other suitable approximation method including, but not limited to, a full Metropolis-Hastings Markov chain Monte Carlo (MCMC) simulation.

The SCNA-fit score for each solution is calculated after optimization of σ_{H}:

$${\hat{σ}}_{H} = {argmax}_{{σ}_{H}} {\sum }_{i = 1}^{N} log L ( {x}_{i} \vert \hat{μ} , {σ}_{i} , {σ}_{H} , θ , {w}_{i} )$$

with the elements of θ calculated for each value of σ_{H} by:

$${\hat{θ}}_{q} = {\sum }_{i = 1}^{N} {w}_{i} \frac{N ( {x}_{i} \vert {\hat{μ}}_{q} , {σ}_{i}^{2} + {σ}_{H}^{2} )}{{L}_{p} ( {x}_{i} \vert \hat{μ} , {σ}_{i} , {σ}_{H} )} ; \\ {\hat{θ}}_{z} = {\sum }_{i = 1}^{N} {w}_{i} \frac{U ( d )}{{L}_{p} ( {x}_{i} \vert \hat{μ} , {σ}_{i} , {σ}_{H} )} \qquad ( 6 )$$

The final calculation of the SCNA-fit log-likelihood for each mode may be obtained by inserting \hat{μ}, \hat{θ} and \hat{σ}_{H} into equation (5). Estimates of the copy-state indicators for each segment are calculated as:

$${\hat{q}}_{i} = P ( {q}_{i} \vert {w}_{i} , λ ) \frac{N ( {x}_{i} \vert {\hat{μ}}_{q} , {σ}_{i}^{2} + {σ}_{H}^{2} )}{L ( {x}_{i} \vert \hat{μ} , {σ}_{i} {\hat{σ}}_{H} , \hat{θ} , {w}_{i} )}$$

$${\hat{z}}_{i} = P ( {z}_{i} \vert {w}_{i} , λ ) \frac{U ( d )}{L ( {x}_{i} \vert \hat{μ} , {σ}_{i} , {\hat{σ}}_{H} , \hat{θ} , {w}_{i} )} ,$$

where each \hat{q}ᵢ is a vector representing the posterior probability of each QεQ integer copy-states, corresponding to the copy-ratios (locations) μ.

Genome-wide cellular copy-profiles may be over-determined with respect to DNA ploidy estimates. An alternate estimate of ploidy may be calculated as the expected absolute copy-number over the genome:

$${\hat{τ}}_{g} = {\sum }_{i = 1}^{N} ( {w}_{i} {\sum }_{q = 0}^{Q - 1} q {\hat{q}}_{iq} ) \qquad ( 7 )$$

By definition, the quantity (\hat{τ}_{g}) is an alternate estimate of cancer ploidy (note an additional factor of 2 is added when HSCRs are used). Because \hat{τ}_{g} is a weighted average over discrete states in the modeled data, it is expected to be more robust to experimental fluctuations that shift or scale the copy-profile slightly. Note that, for this computation, the were calculated with θ_{z}=0, so that the above expectation is over integer states only.

Two additional transformations of the copy-state locations μ may be used when copy-ratios are measured using microarrays. The first of these transformations accounts for the effect of attenuation with an isothermal adsorption model:

$$g ( x ) = \frac{x ( 1 + \hat{φ} )}{( 1 + x \hat{φ} )}$$

where the value \hat{φ} parameterizes the attenuation response in a given sample. The second transformation is a variance stabilization for microarray data:

$$h ( x ) = arc sinh ( \frac{x {e}^{{σ}_{η}^{2}} - 1}{{σ}_{∈}} ) ,$$

where an σ_{η} and σ^{ε} represent multiplicative and additive noise scales for each microarray. This transformation may be applied to the marker-level data during estimation of the xᵢ values, after which their distribution is approximately normal. The normal mixture component specified in equation (4) then becomes h(xᵢ)=h (g(μ_{q}))+εᵢ and the corresponding likelihood calculations may be performed under these transformations.

As discussed above, in some embodiments, additional information may be used to reliably select one of the purity and ploidy solutions from the set of candidates identified by, for example, fitting the model in equation (4). In a given sample, several combinations of theoretically possible purity, ploidy, and copy number values may map to equivalent copy ratios. Additionally, the presence of subclonal SCNAs may result in a spuriously high ploidy solution with an implausible karyotype receiving a greater SCNA-fit likelihood by over-discretizing the copy profile, allowing their assignment to integer copy-levels.

Some embodiments model common cancer karyotypes by grouping sets according to similarities in their cellular homologous copy-number profiles. This method favors simpler solutions, while preserving the flexibility to identify unexpected karyotypes given sufficient evidence from the copy profile. The karyotype models may be constructed directly from the data in a ‘boot-strapping’ fashion, whereby a subset data with relatively unambiguous profiles (e.g., due to high purity values) is used to initialize the models, iteratively allowing more data to be called, etc. In one implementation, previous cytogenetic characterizations of human cancer may be used to guide this process. In addition to the SCNA-fit likelihood values discussed above, the use of karyotype models enables calculation of a karyotype likelihood for each candidate solution in the set of candidate solutions. The karyotype likelihood fit values reflect the similarity of the corresponding karyotype to models associated with the specified disease of the input sample. Accordingly, spurious candidate solutions may be identified and rejected if they do not match a known karyotype model.

In some embodiments, the combination of both SCNA-fit and karyotype likelihoods favors robust and unambiguous identification of the correct purity and ploidy values in many samples. For example, the selection of a solution implying a less common karyotype requires greater evidence from the SCNA-fit of the copy profile.

Prior knowledge of karyotypes characteristic of a particular disease may be summarized as a mixture of K multivariate multinomial distributions over the integer homologous copy-states (e.g., Q=[0,7]) of each chromosome arm. For a given candidate purity and ploidy solution, the corresponding segmental copy-state indicators for each segment i,\hat{q}ᵢⱼ, are summarized into estimates of the J arm-level homologous copy-numbers, denoted Ĉ. The karyotype log-likelihood score may be calculated as:

$${L}_{k} = ( \hat{C} \vert K ) = log {\sum }_{i = 1}^{K} [ {w}_{i} {\prod }_{j = 1}^{J} {\prod }_{q ∈ Q} {K}_{ijq}^{{\hat{C}}_{jq}} ] \qquad ( 8 )$$

where wᵢ denotes the weight of each mixture component. The karyotype models Kᵢ are J×Q SCNA probability matrices obtained by clustering arm-level homologous copy-states of modeled copy-profiles using the standard expectation-maximization (EM) algorithm for multinomial mixtures. This calculation identifies groups of disease subtypes with similar genomic copy profiles. Note that copy-states for both homologues of each arm are modeled (J=78). Karyotype scores for samples with only total copy-ratio data may be calculated using convolution of the multinomial probabilities for the two homologous chromosomes.

The number of clusters K for each disease may be chosen by minimizing the Bayesian information criterion (BIC) complexity penalty: −2\hat{L}ₖ+KJ log (N), where \hat{L}ₖ indicates the sum of L_{K} values over the N input samples, computed using K clusters. In order to avoid local minima, the EM algorithm may be run multiple (e.g., 25) times for each value of Kε[2,8] with randomized starting points and the best model being retained.

In one implementation, the models were constructed in a semi-automated fashion by seeding with relatively unambiguous copy-profiles. As samples were added, the use of recurrent karyotypes identified correct solutions of additional samples, etc. For example, LOH of chr17 occurs in nearly 100% of ovarian carcinoma samples, allowing the model to learn that solutions implying LOH of chr17 are likely to be correct. In total, in this implementation, models for 14 disease types were created. However, it should be appreciated that any suitable number of models may be used. Diseases with fewer than a certain number (e.g., 40) samples may be omitted from this procedure. In addition, a “master” model may be created by combining called primary cancer profiles and the master model may be used for diseases with no specific karyotype model.

In some embodiments, information about mutations (e.g., allelic fractions) may additionally be used to select a candidate solution from the set of candidate solutions. The addition of somatic mutation allelic fractions may allow for increased sensitivity for samples with few SCNAs. However, although the mutation data may help distinguish genome-doubling ambiguity in purity/ploidy estimation, the addition of mutation data may not inform ambiguities of the type b′=b+2(1−α)/D. Thus, such a combined analysis generally facilitates obtaining higher call-rates using the methods described herein.

In order to facilitate rapid analysis of many cancer samples, in some embodiments, at least one processor may be programmed to automatically identify copy profiles that cannot be reliably called and to classify them into informative failure categories, defined by the following criteria: Define m as the sorted vector of posterior genome-wide copy-state allocations (\hat{θ}), so that \hat{m}₁ represents the greatest element of \hat{θ} (the modal copy-state). In one implementation, the vector \hat{m} was constructed with θ₀ replaced by 0 if θ₀<0.01 and b<0.15, so that germline copy-number variants (CNVs) or regions of inherited homozygosity are not confused with small SCNAs implying very pure samples. The categories are then:

1. non-aberrant: \hat{m}₃<0.001, \hat{m}₂<0.005, \hat{σ}<0.02

2. insufficient purity: \hat{m}₃, <0.001, \hat{m}₂<0.005, \hat{σ}≧0.02

3. polygenomic: \hat{θ}_{z}>0.2

These criteria may be applied to the top-ranked mode for each sample (e.g., using combined SCNA-fit and karyotype scores). The use of somatic mutation data may further increase the calling sensitivity within these sample categories.

A framework for calculation of statistical power for the detection of mutations is now discussed. By determining the power to detect a variant, or alternatively, determining a minimum number of reads such that the sensitivity for detecting the mutation is higher than a threshold value, a faster and less-expensive method to detect mutations is arrived at compared to conventional methods, which require a larger amount of DNA and do not rely on estimates of purity, ploidy, and copy number per cell. In some embodiments, using whole-exome sequencing for determining the power to detect a variant depends on the allelic fraction f and local depth of coverage n. To calculate power, the idealized scenario is modeled in which random sequencing errors occur uniformly with rate ε. A minimum number of supporting reads k is determined such that the probability of observing k or more identical non-reference reads due to sequencing error is less than a defined false-positive rate (FPR):

$$k = {argmin}_{m} \vert P ( m ) \leq FPR , \\ where$$

$$P ( m ) = \{ \begin{matrix}1 & if m = 0 \\ 1 - {\sum }_{i = 0}^{m - 1} Binom ( i \vert n , ∈ / 3 ) & if m \geq 1\end{matrix} \}$$

Variants with ≧k supporting reads may then be considered detected. In one implementation, the sequencing error rate ε=1×10⁻³ and FPR=5×10⁻⁷ may be specified for all computations. Power is then calculated as:

$$Pow ( n , f ) = 1 - {\sum }_{i = 0}^{k - 1} Binom ( i \vert n + f ) + d Binom ( k \vert n , f ) , \\ where \\ d = \frac{FPR - P ( k )}{P ( k - 1 ) - P ( k )} . \qquad ( 9 )$$

The case of detecting clonal somatic variants present at a single copy per cancer cell in cancer-tissue derived DNA samples is now considered. Given estimates of purity (α) and local cellular copy-number (qₜ), the allelic fraction of such variants is:

$$δ = \frac{α}{2 ( 1 - α ) + α {q}_{t}} \qquad ( 10 )$$

Power may be calculated in such cases as Pow(n,δ).

A probabilistic model for inference of the integer multiplicities for both germline and somatic point mutation multiplicities and the model may be based on knowledge of purity and genome-wide absolute copy-numbers. When the absolute homologous copy-numbers at a mutant locus is denoted as q₁ and q₂, with q₁≦q₂. The possible multiplicities of germline variants are then:

g_{q}={q₁,q₂,qₜ}

where qₜ=q₁+q₂. Under the assumption that all somatic point-mutations arise uniquely on a single haplotype, the possible multiplicities are:

s_{q}={1, . . . ,q₂}.

Note that when only total copy-ratio data are available, q₂ is unknown, and qₜ may be used instead.

Germline mutations are generally present in both cancer and normal cell populations, with somatic copy-number alterations affecting the allelic fraction. A heterozygous variant in the germline, with multiplicity g_{q} in the cancer genome, has allelic fraction:

$${f}_{{g}_{q }} = \frac{( 1 - α ) + α {g}_{q}}{2 ( 1 - α ) + α {q}_{t}} , \qquad ( 11 )$$

whereas the allelic fraction of homozygous germline variants is 1 regardless of α. For somatic point mutations, the expected allelic fraction at multiplicity s_{q} is f_{sq}=s_{q}δ, with δ as in equation (10).

For example, consider an observed somatic point-mutation of unknown copy s_{q}εs_{q}, observed allelic fraction \hat{f}, and with n total reads covering the locus. The complete likelihood of \hat{f} may be represented as a mixture of Beta distributions corresponding to each element of s_{q}, plus an additional component S corresponding to subclonal states:

$${L}_{m} ( \hat{f} \vert n , {s}_{q} , {w}_{q} , {w}_{{s}_{c}} ) = {\sum }_{{s}_{q} ∈ {s}_{q}} [ {w}_{{s}_{q}} Beta ( {f}_{{s}_{q}} \vert n \hat{f} + 1 , n ( 1 - \hat{f} ) + 1 ) ] + {w}_{{s}_{c}} S ( \hat{f} \vert n , λ ) \qquad ( 12 )$$

where wₛ_{_{q}}εw_{q} specify mixture weights for each state in s_{q} and wₛ_{_{c}} specifies the subclonal component weight. The subclonal component S is specified by composing a Beta distribution (modeling sampling noise) with an exponential distribution over subclonal cancer-cell fractions, having a single parameter λ:

S(\hat{f}|n,λ)=∫₀¹Beta(f|n\hat{f}+1,n(1−\hat{f})+1)Exp(f/δ/λ)δ⁻¹df

Note the change of coordinates in the exponential component using δ; this allows modeling in consistent units of cancer-cell fractions, regardless of purity and local copy-number (note this distribution is renormalized on the unit interval). The probability of a given integer copy-state s_{q} may then be calculated as:

$${\hat{s}}_{q} = \frac{{w}_{{s}_{q}}}{{q}_{2}} \frac{Beta ( {f}_{{s}_{q}} \vert n \hat{f} + 1 , n ( 1 - \hat{f} ) + 1 )}{{L}_{m} ( \hat{f} \vert n , {s}_{q} , {w}_{q} , {w}_{{s}_{c}} )}$$

Similarly, the probability that a given mutation is subclonal may be calculated as:

$${\hat{s}}_{c} = {w}_{{s}_{c}} {q}_{2} \frac{S ( \hat{f} \vert n , λ )}{{L}_{m} ( \hat{f} \vert n , {s}_{q} , {w}_{q} , {w}_{{s}_{c}} )}$$

Optimization of mixture components weights corresponding to integer somatic multiplicities may be accomplished in a manner similar to that described for the SNCA mixture model in equation (6). A Dirichlet prior may be specified as a vector of pseudo-counts equivalent to prior observations of each multiplicity value. Weights are then calculated as the mode of the posterior Dirichlet calculated from the observed counts. These computations may be used to calculate a mutation-score likelihood for each purity/ploidy solution when paired SCNA and somatic point mutation data are used.

In the above description, an exemplary formulation of determining copy number per cell (such as cancer cell) and point mutation multiplicity information, in accordance with some embodiments of the invention, has been described. The methods and apparatus described herein were used in two applications, described in more detail below, to demonstrate the effectiveness and potential of these techniques in providing a better understanding of the cancer genome. In the first example, cellular copy-number alterations in a large group of cancer samples were analyzed to evaluate common characteristics across disease types. In the second example, samples associated with a particular disease type (Chronic lymphocytic leukemia (CLL)) were analyzed to evaluate the mechanisms of cancer evolution and the impact of cancer evolution on therapy. It should be appreciated, however, that although only two examples are described, the methods and apparatus described herein may be used in any suitable way to analyze characteristics of the cancer genome and embodiments of the invention are not limited in this respect.

### Example 1

### Cellular Copy-Number Alterations in Cancer

The methods described herein were used to perform a large-scale ‘pan-cancer’ analysis of copy-number alterations on a cellular basis, across 3,155 cancer samples, representing 25 diseases with at least 20 samples each. The analysis revealed that whole-genome doubling events occur frequently during tumorigenesis, ultimately resulting in mature cancers descended from doubled cells bearing complex karyotypes. Despite evidence that genome doublings can result in genetic instability and accelerate oncogenesis, the incidence and timing of such events had not been broadly characterized in human cancer.

Estimates of purity and copy number per cancer cell enabled the analysis of allelic-fraction values (the fraction of non-reference sequencing reads supporting a mutation) to distinguish clonal and subclonal point mutations, and to detect macroscopic subclonal structure in an ovarian cancer sample. Clonal events may be classified as homozygous or heterozygous in the cancer cells, guiding interpretation of their function. In addition, the ability to quantify integer multiplicity of mutations aided in the relative timing of segmental DNA copy-number gains, as multiplicity values of greater than one imply that the point mutation preceded copy gain of the locus. Controlling for purity and local copy-number allow such timings to be calculated more generally than in the special case of copy-neutral loss of heterozygosity. Furthermore, the data allowed for characterization of somatic cancer evolution with respect to whole-genome doubling, as demonstrated in ovarian carcinoma and associated with clinicopathological values.

Estimation of Purity and Ploidy Across Cancer Types

The methods described herein were used to analyze allelic copy-ratio profiles derived from SNP arrays from 3,155 cancer samples, comprising 2,791 tissue specimens and 364 cancer cell lines. This yielded predicted purity and ploidy values and the segmented absolute allelic copy number of each tumor sample analyzed.

The samples came from two TCGA pilot studies describing glioblastoma multiforme (GBM; 192 samples) and ovarian carcinoma (488 samples), as well as 2,445 profiles incorporated from a previous pan-cancer copy-number analysis. A minority of these samples (519 or 16.4%) could not be analyzed because they lacked clearly identifiable SCNAs, either because they were nearly euploid (nonaberrant), or were excessively contaminated with normal cells (insufficient purity). Although sequencing data for somatic point mutations may have resolved these cases, such data were not available for the majority of samples in this cohort.

For the 2,636 samples with detectable SCNAs, purity and ploidy calls were determined using the methods described herein for 92% of cases, and the remaining samples were designated as ‘polygenomic’ (genomically heterogeneous). The fraction of called samples varied by disease type, from 34.6% (myeloproliferative disease; mostly nonaberrant genomes) to 96.7% (ovarian carcinoma; 100% aberrant genomes), with a median call-rate of 79.2%.

The distributions of estimated purity varied among cancer types, with the tested lung, esophageal and breast cancer samples being the least pure on average in the data set. The effect of contamination was readily visible in the copy ratios of impure cancer types. Distributions of estimated ploidy were qualitatively consistent with those derived from previously obtained cytological data for each cancer type.

Power for Detection of Somatic Point-Mutations by Sequencing

Both sample purity and ploidy affect the local depth of sequencing necessary to detect point mutations. For example, suppose that a region is present at six copies with only one copy carrying a mutation in a sample that has 50% contamination with normal cells. In this case, only one of eight alleles at this locus (six from the cancer cells and two from the normal cells) carry the mutation. It is therefore expected that the mutation will be observed in only 12.5% of reads. Given this allelic fraction, local sequence coverage of 33-fold is required to detect the mutation with 80% sensitivity, assuming a sequencing error rate of 10⁻³ per base and a false-positive rate controlled at <5×10⁻⁷.

Using the estimates of purity and genome-wide integer copy numbers obtained in accordance with the methods described herein, the required coverage for powered detection of mutations present at specified allelic multiplicity per cancer cell was determined. Similar considerations apply to detecting subclonal mutations, present in a fraction of cancer cells, by using fractional multiplicities. It is noted that consideration of sample purity in units of cells, rather than DNA fraction, is preferred for devising power calculations for sequencing experiments, because many somatic alterations of interest are expected to occur at a single copy per cancer cell.

The distribution of purity and ploidy values in cancer samples analyzed for allelic copy number was analyzed to determine an appropriate depth of sequencing coverage needed to detect clonal mutations with power 0.8 in each sample. For this purpose, the number of reads needed to detect a mutation present in one copy, at a locus present at the average copy number, given the sample's purity was calculated. Alternatively, a particular percentile on the copy-number distribution could have been chosen. For such a locus, it was found that 30×local coverage would suffice for most samples. By contrast, a locus of average copy number with a mutation carried in a subclone at 0.2 cancer-cell fraction would require coverage of ˜100-fold to allow detection in about half of the samples. Using these calculations and the distribution of local coverage along the genome (which depends on the specific sequencing technology), one can determine the average coverage necessary to obtain sufficient power in a predefined fraction of the genome (e.g., >80% power in >80% of the genome).

Next, whole-exome sequencing data (˜150×average coverage) was examined from 214 TCGA ovarian carcinoma samples to determine whether detection power was related to the number of mutations actually observed. For each sample, the proportion of loci for which the local coverage provided at least 80% power to detect mutations present at single copy in a subclone present at 0.05 cancer-cell fraction was calculated. The samples with the lowest proportion of such well-powered loci tended to be those in which the fewest such mutations were detected, suggesting that the failure to find such mutations was due to the lack of power. This result also demonstrates the importance of power calculations for characterization of the subclonal frequency spectrum.

Multiplicity Analysis of Somatic Point-Mutations

The methods describe herein were further used to convert the allelic fraction of mutations to cellular multiplicity estimates. For this purpose, 29,268 somatic mutations identified in whole-exome hybrid capture Illumina sequencing data from 214 ovarian carcinoma-normal pairs were analyzed. Purity, ploidy and absolute copy-number values were obtained from Affymetrix SNP6.0 hybridization data on the same DNA aliquot that was sequenced, allowing the rescaling of allelic fractions to units of multiplicity.

This procedure identified pervasive subclonal point-mutations in ovarian carcinoma samples. Although many of the mutations were clustered around integer multiplicities, a substantial fraction occurred at multiplicities substantially less than one copy per average cancer cell, consistent with subclonal multiplicity.

Several lines of evidence support the validity of these subclonal mutations, including Illumina resequencing of an independent whole-genome amplification aliquot, which confirmed both their presence, and that their allelic fractions corresponded to subclonal multiplicity values. In addition, the mutation spectrum seen for clonal and subclonal mutations was similar, consistent with a common mechanism of origin. Power calculations showed that these samples were at least 80% powered for detection of subclonal mutations occurring in cancer-cell fractions ranging from 0.1 to 0.53, with a median of 0.19.

The distribution of subclonal multiplicity was similar in the majority of samples; it rapidly increased at the sample-specific detection limit and then decreased in a manner approximated by an exponential decay in the multiplicity range of 0.05 to 0.5 when pooling across all samples. In contrast, the high-grade serous ovarian carcinoma (HGS-OvCa) sample TCGA-24-1603 showed evidence for discrete ‘macroscopic subclones’. Rescaling of subclonal SCNAs and point mutations to units of cancer cell fraction revealed discrete clusters near fractions 0.2, 0.3 and 0.6, implying the alterations within each cluster likely co-occurred in the same cancer cells. This combination of cell fractions sums to more than one, implying that at least one of the detected subclones was nested inside another.

The methods described herein were then used to analyze the multiplicity of both the reference and alternate alleles in order to classify point mutations as either heterozygous or homozygous in the affected cell fraction. Fifteen genes with mutations recently identified in these data were considered, including five known tumor suppressor genes and five oncogenes. The frequency of homozygous mutations in known tumor suppressor genes and oncogenes was significantly different, with a significantly elevated fraction of homozygous mutations in the tumor suppressor gene and no homozygous mutations in the oncogenes. This result provides evidence supporting CDK12 as a candidate tumor suppressor gene in ovarian carcinoma, since 7 of 12 CDK12 mutations were homozygous.

Overall, TP53 had among the greatest fraction of clonal, homozygous and ‘multiplicity >1’ mutations of any gene in the coding exome, demonstrating the clear identification of a key initiating event in HGS-OvCa carcinogenesis directly from multiplicity analysis.

Whole-Genome Doubling Occurs Frequently in Human Cancer

For many cancer types, the distribution of total copy number (ploidy) was markedly bi-modal, consistent with chromosome-count profiles derived from SKY. Although these results are consistent with whole-genome doubling during their somatic evolution, it has been difficult to rule out the alternative hypothesis that evolution of high-ploidy karyotypes results from a process of successive partial amplifications.

To study genome doublings, homologous copy-number information—that is, the copy numbers, bᵢ and cᵢ, of the two homologous chromosome segments at each locus were used. By looking at the distributions of bᵢ and cᵢ across the genome, inferences could be drawn regarding genome doubling. Immediately following genome doubling, both bᵢ and cᵢ would be even numbers. Following the loss of a single copy of a region, the larger of bᵢ and cᵢ will remain even, but the smaller would become odd. In fact, when high-ploidy samples were examined, it was discerned that the higher of bᵢ and ci was usually even throughout the genome, consistent with their having arisen by doubling of the entire genome. Using simulations, it was found that the observed profiles were unlikely to arise owing to SCNAs occurring in serial fashion at multiple independent chromosomes.

Using such information, samples were classified into three groups, which were interpreted as corresponding to 0, 1 and >1 genome doubling events in the clonal evolution of the cancer. These three groups had modal ploidy values of 1.75, 2.75 and 4.0, respectively, and also segregated into three clusters by ploidy and mean homologous copy-number imbalance. This was interpreted as evidence of SNCAs occurring with net losses, interspersed with the genome doublings. This process resulted in intermediate ploidy values for the doubled clones (2.2-3.4n), with pervasive imbalance of homologous chromosomes.

The frequency of genome doubling varied across cancer types, reflecting differences in disease-specific biology and clinical progression status. Hematopoietic neoplasms (myeloproliferative disease, acute lymphoblastic leukemia) had nearly no doubling events, whereas glioblastoma multiforme, renal cell carcinoma, prostate cancer, various sarcomas, hepatocellular carcinoma and medulloblastoma all had ˜25% incidence of doubling.

Genome doubling was more common in epithelial cancers, with colorectal, breast, lung, ovarian and esophageal cancers all having >50% incidence of doubling. Esophageal adenocarcinoma had the greatest doubling incidence, consistent with previous reports of frequent 4n populations at various stages of Barrett's esophagus progression.

Specific Aneuploidies Precede Genome Doubling

The methods described herein were also used to infer the temporal order of genome doubling in tumorigenesis, relative to SNCAs involving specific chromosome arms. In many cancer types, the fixation of arm-level SCNAs was inferred to occur before genome doubling, because both doubled and nondoubled samples had similar frequencies of specific arm-level SNCAs.

In glioblastoma multiforme samples, loss of heterozygosity involving chromosomes 9 and 10, and gains of chromosome 7 occurred at equivalent frequencies, demonstrating that the most common broad SCNAs in glioblastoma multiforme occur before genome doubling. Gain of chromosomes 19 and 20 was nearly exclusive to nondoubled samples, and several arms had greater frequency of loss of heterozygosity in doubled samples, suggesting that additional biological differences underlie these samples.

Cases with ploidy 2N and 4N with no observed SCNAs were discarded from the analysis. For many cancer types, such cases were rare, due to the tendency for chromosomal losses after doubling. The representation of specific cancer subtypes may be biased by differences in ascertainment, however.

In contrast to broad chromosomal alterations, focal SCNA events occurred at greater frequency in doubled genomes. Consistent with previous reports, the observed frequency of focal SCNAs as a function of their length (L) followed power-law scaling: P(L)∝L−α, for L>0.5 Mb. Genome doubling was associated with a larger overall number of SCNAs; however, estimates of a near 1 were obtained for each group, suggesting that the mechanism(s) by which they were generated did not greatly depend on ploidy.

Genome Doubling Influences Progression of Ovarian Carcinoma

Whole-genome doubling occurrence in high-grade serous ovarian carcinoma was correlated with other genetic and clinical features. Genome-doubled samples showed a higher incidence of heterozygous mutations, but correcting for sample ploidy removed this effect, suggesting that the per-base mutation rates are equivalent. Clonal mutations at multiplicity >1 were approximately tenfold more prevalent in doubled samples; many of these events likely occurred before the doubling event. Genome-doubled samples had significantly lower frequencies of both of homozygous deletions and of clonal homozygous mutations. It is expected that many of the observed homozygous alterations in the doubled samples were fixed before genome doubling.

The lower incidence of homozygous mutations in genome-doubled samples may reflect the fact that more events are required to render a mutation homozygous in a genome-doubled sample (although the effect may be partially offset by a possible increase in genetic instability following doubling, for example, by centrosome duplication). These considerations suggest that genome-doubled samples evolve by means of distinct trajectories, because inactivation of tumor suppressors may occur less frequently after doubling.

It was noted that 13 of the 15 detected point mutations in the tumor suppressor NF 1 occurred in the 93 ovarian samples that had not undergone genome doubling, and these mutations were uniformly homozygous. This is consistent with selection for recessive inactivation of NF1, a typical pattern for a tumor suppressor gene. It also suggests that nongenome-doubled ovarian carcinoma samples evolved through a distinct trajectory, rather than being precursors to doubled samples. If not, many NF1 mutations would be homozygous with multiplicity >1 in doubled samples, as is seen for TP53.

It was also noted that genome-doubled samples were associated with a significant increase in the age at pathological diagnosis and with a significantly greater incidence of cancer recurrence.

Analysis of SCNAs using methods described herein demonstrated that many of the copy-number alterations analyzed were fixed in the cancer lineage represented in the sample. This was recapitulated in ovarian cancer by somatic point-mutations, many of which were fixed at integer multiplicity. Classification of point mutations based on their multiplicities may help distinguish tumor suppressors and oncogenes. Knowledge of discrete copy-states, subclonal structure and genome doubling status provides a foundation for further reconstruction of the phylogenetic relationships within a cancer and the temporal sequence by which a given cancer genome arose.

The methods described herein provide a tool for the design of studies using genomic sequencing to detect variant alleles in cancer tissue samples, based on calculation of sensitivity to detect mutations as a function of sample purity, local copy number and sequencing depth. The high accuracy of purity and ploidy estimates produced by the methods described herein, based on SNP microarray data, makes it possible to determine the sequencing depth required for a given sample or to select suitable samples given a fixed sequencing depth. Such considerations are vital to the interpretation of subclonal point-mutations.

Analysis of the predicted absolute allelic copy-number profiles across human cancers produced by methods described herein shed new light on cancer genome evolution. The observed SCNA profiles were consistent with a common trajectory consisting of an early period of chromosomal instability followed by the emergence of a stable aneuploid clone, as previously described. The data further indicate that genome doublings occur in a subset of cancer cells already harboring arm-level SCNAs characteristic of the corresponding cancer type. The genomes of these cancers were therefore shaped by selection at chromosomal arm-level resolution before doubling and further clonal outgrowth.

These findings are broadly consistent with an earlier interpretation of primary breast cancer FACS/SKY profiles, and has recently been recapitulated in studies of macro-dissected and ploidy-sorted cell populations, and single-cell sequencing of primary breast tumors. This model represents a departure from the idea that tetraploidization is an initiating event. In addition, the association of genome doubling with epithelial lineage and with age at diagnosis in ovarian carcinoma is consistent with a recently described mechanism linking telomere crisis, DNA damage response, and genome doubling in cultured mouse embryonic fibroblasts.

The analysis of clonality described herein offers a path forward for clinical sequencing of cancer, and provides the means to address recently reported concerns regarding intratumor heterogeneity. Analysis using the methods described herein can identify alterations present in all cancer cells contributing to the DNA aliquot, even if such clonal alterations correspond to the minority of observed mutations. Such alterations are candidate founding oncogenic drivers for a given cancer, which may be the preferred therapeutic targets. Further characterization of subclonal somatic alterations in cancer may become important for understanding variable response to targeted therapeutics, with the clonality of targeted mutations potentially affecting response level.

### Example 2

### Evaluation of Clonal and Subclonal Point Mutations in Cancer

The existence of subclones within cancers has been long appreciated. However, the mutational spectrum of subclones, the hierarchy and temporal order of driving genetic events, and the impact of clonal evolution on clinical course are not well understood. To address these questions, the frequency and evolution of subclonal and clonal mutations in 149 patients with chronic lymphocytic leukemia (CLL) using whole-exome sequencing and somatic copy number alterations in accordance with the methods described herein were analyzed. It was found that 13q deletion, MYD88 mutations, Trisomy 12 were almost always clonal, suggesting that these are the most common initiating genetic events. However, other mutations—such as 11q deletion, SF3B1 and TP53 mutations—were often subclonal and thus likely to arise after the initiating events. To directly observe the evolution of specific mutations, paired longitudinal samples from 18 patients were studied and it was found that 7 patient leukemias displayed significant evolution. Consistent with single timepoint analysis, conversion of subclonal mutations in 11q, SF3B1 and TP53 to clonality over time was observed. An analysis of clinical course revealed that detection of a subclonal driver mutation was associated with earlier therapy (indicative of more rapid progression)—a finding that was validated in the larger cohort of 149 CLL patients. The analysis of subclonal events from whole-exome sequencing data thus contributes to a basic understanding of cancer progression and provides a potential approach for earlier prognosis and therapeutic decision-making Chronic lymphocytic leukemia (CLL) is a common slow-growing B cell malignancy.

A hallmark of this common and incurable leukemia is that CLL patients demonstrate a highly variable clinical course. Recent studies using massively parallel sequencing of whole-genomes have revealed that subpopulations of cells with considerable genetic heterogeneity are present across diverse cancer types, although these analyses have been limited to small cohorts. It was hypothesized that the composition of genetic alterations acquired by subpopulations of CLL cells and the dynamic relationship between subpopulations could affect the tempo of patients' clinical course and response to therapy.

To study the rules of cancer evolution in CLL, an analytic approach that could systematically identify subpopulations within each leukemia sample based on presence of somatic genetic alterations was used. From 149 matched tumor-germline samples, coding somatic single nucleotide variations (SSNVs) were comprehensively identified using whole-exome sequencing (WES). This affordable technology has enabled unbiased genetic characterization of large sets of cancers and its sequencing coverage depth (˜150×) provides adequate power for detecting SSNVs present in as few as 10% of cancer cells, including coding alterations most likely to affect fitness. Thus, allelic fraction of SSNVs was measured as the ratio of the alternate reads to the total number of reads in WES. WES allelic fraction measurements were concordant with deep sequencing and RNA-sequencing (RNAseq) measurements. The methods described herein were used to correct these ‘raw’ allelic fractions for variations in sample purity (i.e., contamination with non-cancer cells), local ploidy (e.g., a SSNV in TP53 can occur in a sample that also has del(17p)) and confidence in the estimation of allelic frequencies (i.e., based on local depth of coverage). This analysis was used to classify a mutation as clonal (or near-clonal) if there was more than a 50% chance that its cancer cell fraction (CCF) was greater than 95% and subclonal otherwise. Overall, 1543 clonal mutations (54% of all detected mutations) and 1266 subclonal mutations (46%) were identified, which corresponded to an average of 10.3±5.5 clonal mutations and 8.5±5.8 subclonal mutations per CLL sample. Only in 3/149 samples were no subclonal SSNVs detected.

Clonal SSNVs involve all leukemic cells, and therefore represent a mixture of genetic events that include initiating driver mutations, passenger mutations acquired before and after the initiating driver event, and driving events that drove subsequent waves of expansion of subpopulations and became fixed in the cellular population. Subclonal SSNVs exist in a fraction of the leukemic population, and thus, are later events in the cancer's evolutionary process. In this example, several questions in the analysis were addressed that relate to the salient clinical characteristics of CLL. As CLL is generally a disease of the elderly, it was asked what impact age at the onset of transformation would have on the diversity of subclones and convergence to clonality. Additionally, it was asked whether a burst of mutagenesis, such as through the process of somatic hypermutation that is inherent to B cell biology, could alter the subclone structure. It was observed that older age at diagnosis and mutated IGHV status (associated with previous AID activity) were both associated with higher numbers of clonal, but not subclonal mutations. These findings provided direct evidence that long-lived genetic alterations in CLL are accumulated early on—likely in the founder cell prior to transformation.

It was also suspected that antileukemia treatment could impact the acquisition of later events in CLL sample and hence the clonal diversity. In support of this, it was observed that CLL samples from patients treated prior to sampling with chemoimmunotherapy contained a higher number of subclonal mutations compared to patients treated after sampling, with similar numbers of clonal mutations. The higher number of subclonal (later) events may testify to a strong extrinsic selection pressure promoting the expansion of subclones to above our detection threshold (CCF of ˜10%).

If subclones are coming up as a consequence of treatment, then it follows that driver mutations could be contributing to the expansion. Thus, the relative enrichment for driver events between the two sets of mutations (subclonal and clonal) was examined across all cancers. While the mutational spectra were highly similar in both groups of mutations, the subclonal set appeared to be enriched with putative driver characteristics. It exhibited a higher proportion of non-synonymous SSNVs in genes included in the cancer gene census and a higher proportion of SSNVs resulting in missense mutations at highly conserved sites. Thus, subclonal mutations are enriched with drivers compared with the clonal mutations. It is important to note that this comparison likely underestimates the relative enrichment, as the clonal set includes founder events (early events) as well as fit late events that at the time of sampling already expanded to involve the entire leukemic population.

It was examined whether different genetic alterations have different roles along the evolutionary history of CLL. In order to accomplish this, a significance analysis for frequent SSNVs and recurrent SCNAs was performed to define a list of CLL driver events. The CCF of these CLL driver events varied substantially across the samples suggesting that they are ‘initiating’ driver events. On the other hand, some alterations (e.g ATM, TP53 and SF3B1) affected generally lower fractions of leukemia cells, suggesting that they were acquired in a subset of cells, and hence later in leukemic development (i.e. ‘progression’ driver events). Thus, in CLL samples that harbored one of the ‘initiating’ events and at least one other CLL driver, the additional event(s) were found at either similar or lower CCF compared to the initiators (data for MYD88 and trisomy 12 shown in (FIG. 2C). This analysis of allelic fraction thus could distinguish between early and later events in CLL evolution.

To directly examine genetic evolution in CLL, CCF for each somatic alteration was compared across two timepoints for 18 of 149 samples for which tumor DNA and RNA from a second timepoint was available (median years between timepoints was 3.45; range 2.4-5). Six patients did not receive treatment throughout the period of study. At timepoint 1, 7 of 12 were chemotherapy-naïve, while 5 of 12 had received prior cytotoxic therapy. Greater than 90% of mutations (either clonal or subclonal) affected an unchanging proportion of leukemia cells over time. On the other hand, 34 SSNVs [8.5%] and 6 SCNAs [13%] affected a relatively smaller subclone (or were not detected at timepoint 1), and subsequently expanded to involve most of the leukemia population (CCF greater than 0.7, q<0.1 for significant change in CCF). These expanding SSNVs were enriched in genes in the cancer gene census, and in CLL drivers, and suggest that these mutations not only mark genetic evolution but also provide a fitness advantage. Marked evolution was observed in 7 of 18 sample sets—all from patients who had received intervening treatment and none from continuously untreated patients. Sample sets with evidence of clonal evolution were all associated with the poor prognostic marker, unmutated IGHV status. Evolution followed one of two patterns: (i) a progeny clone expanded and replaced the parent clone (linear evolution), or (ii) A progeny clone replaced a sibling clone (branched evolution). Both patterns of evolution were reproduced in 6 of 6 sample pairs that underwent targeted re-sequencing to deep coverage. Thus, samples requiring treatment demonstrated evident genetic evolution which was marked by emergence of subclones of increased fitness, while samples lacking need for treatment maintained a relatively stable balance between clonal and subclonal compartments over time.

The changes in the genetic composition of CLL cells were transcribed and propagated through the cellular network to affect the leukemic phenotype. Importantly, however clonal evolution was associated with shortened time to retreatment, similar to trends noted in SCNA-based studies. At least one ‘CLL driver’ in the expanding subclones was identifiable in 6 of 7 evolved CLL samples. In 4 of these 6 CLL the expanding CLL driver was detectable by WES already in the pretreatment sample and explain the graph (it was found that the presence of a subclonal driver is an indicator of worse outcome, specifically with treatment).

These results stimulated an examination of the question of whether the characteristics of the subclonal population—in particular, presence of a CLL driver—could have impact on subsequent clinical outcome. Out of the set of 149 samples, 59 samples were identified with subclones (CCF<0.7) that harbored a CLL driver (or a gene in the cancer gene census with a nonsilent mutation in a highly conserved site). Compared to the 90 samples with subclones lacking CLL drivers, patient samples with subclonal CLL drivers demonstrated a shorter time from diagnosis to first therapy, and between sampling and treatment. For the 67 patients who were treated after sampling (median time to first therapy from time of samples was 6.4 months (range 0-74), patients requiring retreatment had nearly two fold more subclonal drivers compared with CLLs from patients not requiring treatment, while the number of clonal drivers was similar. Furthermore, a Kaplan Meier analysis demonstrated that patients whose leukemia cells contained detectable pre-treatment subclonal drivers required retreatment earlier, indicating a more rapid disease course. Analysis using a Cox regression model of standard CLL prognostic factors (IGHV status, prior therapy and high risk cytogenetics) demonstrated that the presence of a subclonal driver is a risk factor for earlier retreatment. Notably, the presence of a driver irrespective of its CCF failed to identify patients with a shorter time to retreatment. Thus, the detection of subclonal drivers (a testimony to an active evolutionary process), is associated with worse clinical outcome, and shorter duration of therapeutic effect. The results of this example adds to an understanding of CLL biology. Distinct periods in the history of CLL were traced, where the composition of mutations is guided by differences in quality and intensity of the selective pressures. A discrete set of early somatic alterations (del(13q.13.4), MYD88, trisomy 12) were also identified. These are found predominately in B cell malignancies, and involve the exploitation of physiologic B cell pathways. Such alterations comprise ideal initial transforming events, allowing evasion of oncogene-induced senescence triggered for example by RAS family activation, which appeared as later events in CLL.

From the therapeutic perspective, it was shown that subclonal drivers that will later become dominant, can already be detected prior to therapy. Hence, analysis of a pre-treatment sample using the methods described herein can be informative regarding the genetic composition of leukemia cells upon relapse, as well as the rapidity with which this relapse will occur. Although conclusive data can only be obtained in controlled trials, the data described in this example suggest at a hastening of the evolutionary process with treatment (increased number of subclonal mutations in post-treatment samples, and lack of evolution in untreated tumors). This supports a potential mechanistic justification for the empirical practice of ‘watch and wait’ as the CLL treatment paradigm. Cytotoxic therapy—by removing the incumbent clone—can act as a ‘mass extinction’ event, and shift the dynamic evolutionary landscape in favor of a more aggressive clone.

In this example, it was demonstrated that cancer evolution may be determined with WES. These innovations will allow characterization of the subclonal mutation spectrum in large, publically available datasets. Additionally, because these techniques may be implemented using a single DNA measurement (WES) at moderate sequence coverage (150×), the implementation described here may be readily adopted for clinical purposes. This new knowledge provides the opportunity to develop novel therapeutic paradigms, to address, in addition to the individual genetic lesions (i.e. ‘targeted therapy’), the evolutionary development of a cancer as well.

### Validation of Longitudinal Analysis Techniques

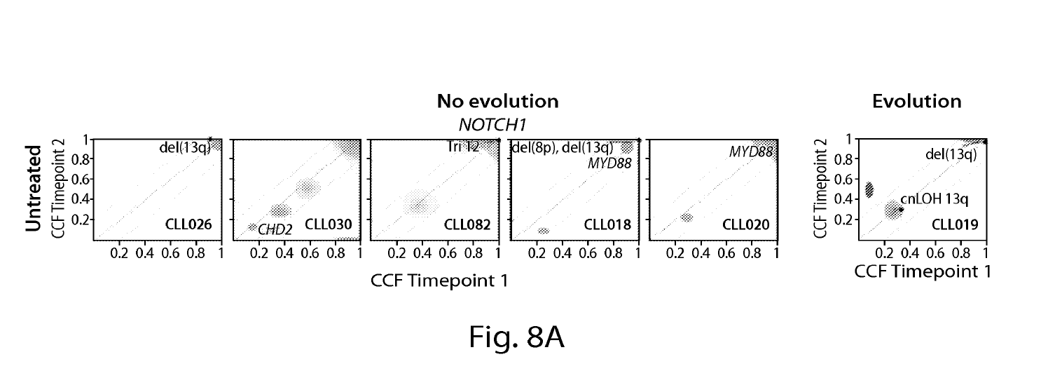

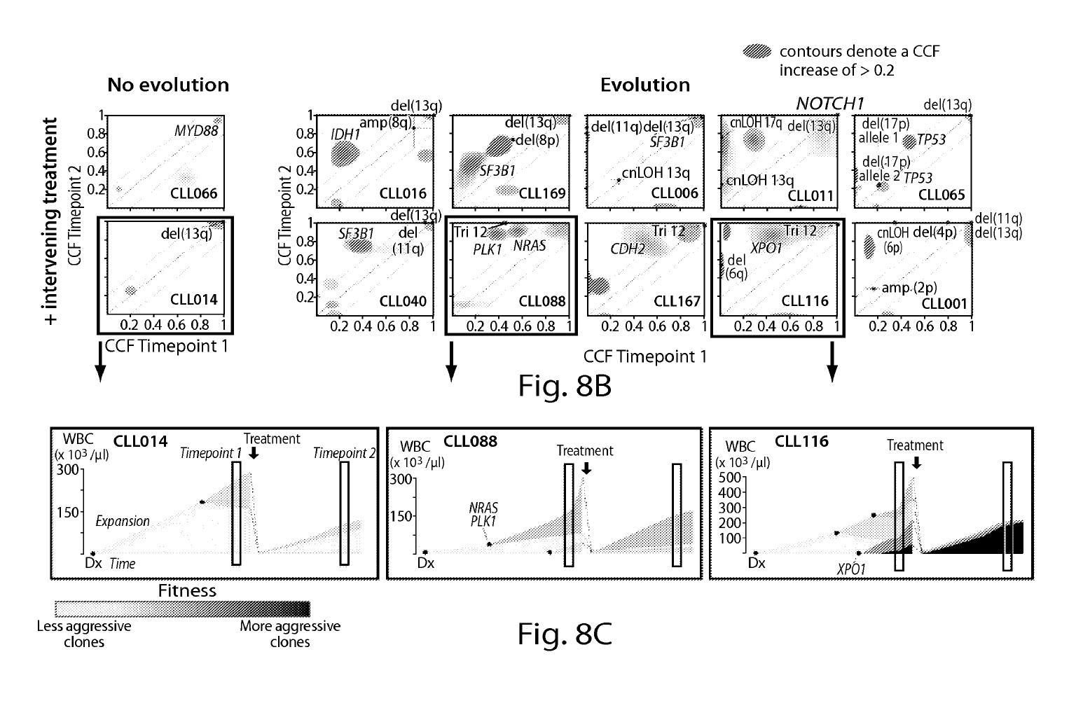

FIGS. 8A-C illustrate the results of a longitudinal analysis of subclonal evolution in CLL and its relation to therapy. These results were published in a paper entitled “Evolution and Impact of Subclonal Mutations in Chronic Lymphocytic Leukemia,” Cell 152, 714-726, Feb. 14, 2013, and provide a validation of the techniques described herein relating to genetic evolution of cancer cells.

FIGS. 8A and 8B illustrate joint distributions of cancer cell fraction (CCF) values across two time points. The dotted diagonal lines represent y=x or where identical CCF values across the two time points fall, and the dotted parallel lines denote the 0.2 CCF interval on either side of the y=x line. Likely driver mutations are also shown in each subgraph. FIG. 8A represents data from six CLL patients having no intervening treatment between the two time points, and FIG. 8B represents data from twelve CLL patients having intervening treatment between the two time points. The CLL patients were classified according to clonal evolution status, based on the presence of mutations with an increase of CCF>0.2 across the two time points. FIG. 8C illustrates a hypothesized sequence of evolution, inferred from the patient's white blood cell counts, treatment dates, and changes in CCF for three representative examples. Further details are available in the published paper, the entire contents of which are incorporated by reference herein, but are not reproduced here for brevity.

### Exemplary Computer System

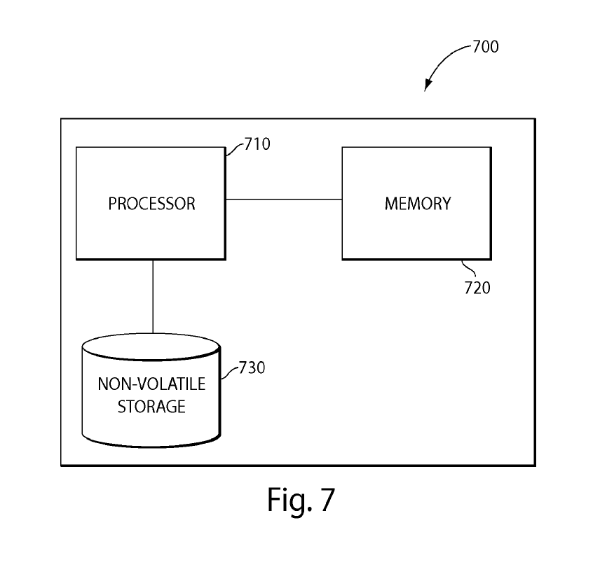

An illustrative implementation of a computer system 700 that may be used in connection with any of the embodiments of the invention described herein is shown in FIG. 7. The computer system 700 may include one or more processors 710 and one or more computer-readable non-transitory storage media (e.g., memory 720 and one or more non-volatile storage media 730). The processor 710 may control writing data to and reading data from the memory 720 and the non-volatile storage device 730 in any suitable manner, as the aspects of the present invention described herein are not limited in this respect. To perform any of the functionality described herein, the processor 710 may execute one or more instructions stored in one or more computer-readable storage media (e.g., the memory 720), which may serve as non-transitory computer-readable storage media storing instructions for execution by the processor 710.

The above-described embodiments of the present invention can be implemented in any of numerous ways. For example, the embodiments may be implemented using hardware, software or a combination thereof. When implemented in software, the software code can be executed on any suitable processor or collection of processors, whether provided in a single computer or distributed among multiple computers. It should be appreciated that any component or collection of components that perform the functions described above can be generically considered as one or more controllers that control the above-discussed functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments of the present invention comprises at least one non-transitory computer-readable storage medium (e.g., a computer memory, a floppy disk, a compact disk, a tape, etc.) encoded with a computer program (i.e., a plurality of instructions), which, when executed on a processor, performs the above-discussed functions of the embodiments of the present invention. The computer-readable storage medium can be transportable such that the program stored thereon can be loaded onto any computer resource to implement the aspects of the present invention discussed herein. In addition, it should be appreciated that the reference to a computer program which, when executed, performs the above-discussed functions, is not limited to an application program running on a host computer. Rather, the term computer program is used herein in a generic sense to reference any type of computer code (e.g., software or microcode) that can be employed to program a processor to implement the above-discussed aspects of the present invention.

Various aspects of the present invention may be used alone, in combination, or in a variety of arrangements not specifically discussed in the embodiments described in the foregoing and are therefore not limited in their application to the details and arrangement of components set forth in the foregoing description or illustrated in the drawings. For example, aspects described in one embodiment may be combined in any manner with aspects described in other embodiments.

Also, embodiments of the invention may be implemented as one or more methods, of which an example has been provided. The acts performed as part of the method(s) may be ordered in any suitable way. Accordingly, embodiments may be constructed in which acts are performed in an order different than illustrated, which may include performing some acts simultaneously, even though shown as sequential acts in illustrative embodiments.

Use of ordinal terms such as “first,” “second,” “third,” etc., in the claims to modify a claim element does not by itself connote any priority, precedence, or order of one claim element over another or the temporal order in which acts of a method are performed. Such terms are used merely as labels to distinguish one claim element having a certain name from another element having a same name (but for use of the ordinal term).

The phraseology and terminology used herein is for the purpose of description and should not be regarded as limiting. The use of “including,” “comprising,” “having,” “containing”, “involving”, and variations thereof, is meant to encompass the items listed thereafter and additional items.

Having described several embodiments of the invention in detail, various modifications and improvements will readily occur to those skilled in the art. Such modifications and improvements are intended to be within the spirit and scope of the invention. Accordingly, the foregoing description is by way of example only, and is not intended as limiting. The invention is limited only as defined by the following claims and the equivalents thereto.

1. A method of determining a copy number per cancer cell in a sample of cells, the method comprising:
receiving a relative copy number profile for DNA segments extracted from the sample;
determining based, at least in part, on the relative copy number profile, a set of candidate solutions by estimating purity and ploidy from first information about somatic copy number alterations in the sample;
determining a likelihood fit score for each of the solutions in the set of candidate solutions, wherein the likelihood fit score is determined based, at least in part, on the first information about somatic copy number alterations in the sample and second information, wherein the second information comprises information about one or more mutations detected in the sample and/or information about karyotype copy profile characteristics of a particular disease;
selecting a solution from the set of candidate solutions based, at least in part, on the likelihood fit score associated with each candidate solution; and
determining the copy number per cancer cell in accordance with the selected solution.

2. The method of claim 1, wherein the sample comprises cancer cells and normal cells.

3. (canceled)

4. The method of claim 1, further comprising using the information about karyotype copy profile characteristics as prior information to reduce a number of likely candidate solutions.

5. The method of claim 1, wherein the one or more mutations comprises one or more point mutations.

6. The method of claim 1, wherein the relative copy profile and/or the information about the one or more mutations is generated using massively parallel sequencing.

7. (canceled)

8. The method of claim 6, wherein using massively parallel sequencing comprises using whole-genome sequencing and/or whole-exome sequencing.

9. (canceled)

10. The method of claim 1, wherein the information about one or more mutations comprises an allelic fraction.

11. (canceled)

12. The method of claim 1, wherein estimating purity and ploidy comprises jointly estimating purity and ploidy.

13. The method of claim 1, further comprising:
classifying a first mutation of the one or more detected mutations as clonal or subclonal, wherein the classification is based, at least in part, on a probability that the first mutation exists in a cancer cell fraction below a threshold value.

14. The method of claim 13, wherein the classifying the first mutation comprises classifying the first mutation as clonal if there is a greater than 50% probability that the first mutation exists in a cancer cell fraction below the threshold value and classifying the first mutation as subclonal otherwise.

15. The method of claim 1, wherein the relative copy number profile represents homologue-specific copy ratio data and/or total-copy ratio data.

16. (canceled)

17. The method of claim 1, wherein determining the copy number per cancer cell comprises determining the copy number per cancer cell at a number of timepoints.

18. The method of claim 1, wherein determining the copy number per cancer cell comprises determining the copy number per cancer cell using a first sample obtained before treatment and determining a copy number per cancer cell using a second sample obtained after treatment.

19. The method of claim 18, further comprising determining a treatment strategy based at least in art, on a comparison of the copy number per cancer cell determined for the first sample obtained before treatment and the copy number per cancer cell determined for the second sample obtained after treatment.

20. A non-transitory computer-readable medium encoded with a plurality of instructions that, when executed by a computer, perform a method, comprising:
receiving a relative copy number profile for DNA segments extracted from the sample;
determining based, at least in part, on the relative copy number profile, a set of candidate solutions by estimating purity and ploidy from information about somatic copy number alterations in the sample;
determining a likelihood fit score for each of the solutions in the set of candidate solutions, wherein the likelihood fit score is determined based, at least in part, on the first information about somatic copy number alterations in the sample and second information, wherein the second information comprises information about one or more mutations detected in the sample and/or information about karyotype copy profile characteristics of a particular disease;
selecting a solution from the set of candidate solutions based, at least in part, on the likelihood fit score associated with each candidate solution; and
determining the copy number per cancer cell in accordance with the selected solution.

21-22. (canceled)

23. The computer-readable medium of claim 20, wherein determining the copy number per cancer cell comprises determining the copy number per cancer cell for a first sample obtained before treatment and determining a copy number per cancer cell for a second sample obtained after treatment.

24. The computer-readable medium of claim 23, further comprising determining a treatment strategy based, at least in part, on a comparison of the copy number per cancer cell determined from the first sample obtained before treatment and the copy number per cancer cell determined from the second sample obtained after treatment.

25. A computer system, comprising:
at least one processor programmed to:
receive a relative copy number profile for DNA segments extracted from a sample;
determine based, at least in part, on the relative copy number profile, a set of candidate solutions by estimating purity and ploidy from information about somatic copy number alterations in the sample;
determine a likelihood fit score for each of the solutions in the set of candidate solutions, wherein the likelihood fit score is determined based, at least in part, on the first information about somatic copy number alterations in the sample and second information, wherein the second information comprises information about one or more mutations detected in the sample and/or information about karyotype copy profile characteristics of a particular disease;
select a solution from the set of candidate solutions based, at least in part, on the likelihood fit score associated with each candidate solution; and
determine the copy number per cancer cell in accordance with the selected solution.

26-27. (canceled)

28. The computer system of claim 25, wherein determining the copy number per cancer cell comprises determining the copy number per cancer cell using a first sample obtained before treatment and determining a copy number per cancer cell using a second sample obtained after treatment.

29. The computer system of claim 28, wherein the at least one processor is further programmed to determine a treatment strategy based, at least in part, on a comparison of the copy number per cancer cell determined for the first sample obtained before treatment and the copy number per cancer cell determined for the second sample obtained after treatment.

30-51. (canceled)
